(19) United States
(12) Patent Application Publication
(10) Pub. No.: US 2017/0057907 A1
(43) Pub. Date: Mar. 2, 2017

(54) METHOD FOR THE REDUCTIVE AMINATION OF ALDEHYDES

(71) Applicant: BASF SE, Ludwigshafen (DE)

(72) Inventors: Christian EIDAMSHAUS, Wiesloch (DE); Ansgar Gereon ALTENHOFF, Heidelberg (DE)

(73) Assignee: BASF SE, Ludwigshafen (DE)

(21) Appl. No.: 15/120,028

(22) PCT Filed: Feb. 2, 2015
(86) PCT No.: PCT/EP2015/052028

§ 371 Date: Aug. 18, 2016

(30) Foreign Application Priority Data
Feb. 19, 2014 (EP) 14155796.7

(51) Int. Cl. C07C 209/26

(52) U.S. Cl. CPC C07C 209/26

(57) ABSTRACT

In a process for effecting reductive amination by reacting a carbonyl compound with a secondary amine and hydrogen, the reaction is carried out as a continuous operation and the reaction mixture comprising the carbonyl compound, the secondary amine and hydrogen is contacted first with an acidic fixed bed and then with a fixed bed of hydrogenation catalyst (hereinafter referred to as a hydrogenation fixed bed).

The invention relates to processes for effecting reductive amination by reacting a carbonyl compound with a secondary amine and hydrogen, wherein the reaction is carried out as a continuous operation and the reaction mixture comprising the carbonyl compound, the secondary amine and hydrogen is contacted first with an acidic fixed bed and with a fixed bed of hydrogenation catalyst (hereinafter referred to as a hydrogenation fixed bed).

Reductive amination is a customary and economically significant process for preparing amines. In reductive amination, ketones or aldehydes are converted into an amine. A known reductive amination process is the reaction of a carbonyl compound with an amine in the presence of hydrogen, as described, for example, in WO 01/05741.

Prior European application (application number 13173233.1, BASF reference PF 75487) still unpublished at the filing date of this application describes the preparation of 2-chlorodimethylbenzylamine by reductive amination, i.e., by reacting 2-chlorobenzaldehyde with an amine and hydrogen.

It is an object of the present invention to further improve the reductive amination process. What is desired is a very simple and economical process which can be carried out as a continuous operation. The yield of amines prepared and the selectivity should be very high.

We have found that this object is achieved by the above-described process.

The Starting Materials

What is concerned is a process in which a carbonyl compound, a secondary amine and hydrogen are co-reacted.

A carbonyl compound is an organic compound having at least one carbonyl group. Preferred carbonyl compounds have one or two carbonyl groups, particularly preferred carbonyl compounds have only one carbonyl group. Suitable carbonyl groups include aldehyde groups and ketone groups. The carbonyl group is preferably an aldehyde group.

Particularly preferred carbonyl compounds are aldehydes, which term is to be understood as meaning an organic compound having one or two aldehyde groups; aldehydes having one aldehyde group as the sole carbonyl group are very particularly preferred.

The carbonyl compound generally has a molecular weight of no more than 1000 g/mol, more particularly no more than 500 g/mol, with a molecular weight of no more than 300 g/mol being particularly preferred. In a very particularly preferred embodiment, a carbonyl compound having a molecular weight of no more than 200 g/mol is used.

The carbonyl compound may be liquid or gaseous at standard conditions (20° C., 1 bar), preferably liquid.

The carbonyl compound preferably comprises an aromatic ring system; more particularly an aldehyde having an aromatic ring system is concerned.

In a preferred embodiment, the aldehyde is an aromatic ring system composed of 5 to 12 carbon atoms and substituted with an organic group having an aldehyde group and optional further substituents selected from organic groups or halogen atoms. Possible further substituents also include organic groups having heteroatoms such as oxygen or nitrogen. Preferably, further substituents are selected exclusively from hydrocarbon groups or halogen atoms. The aforementioned aldehyde preferably comprises no more than two further substituents, more particularly it comprises no further substituents or only one further substituent.

In a particular embodiment, the aldehyde is a benzene ring substituted with an organic group having an aldehyde group, preferably an aliphatic group having an aldehyde group, and a halogen atom, preferably a chlorine atom.

In a particularly preferred embodiment the aldehyde is 2-chlorobenzaldehyde of formula I

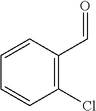

The secondary amine may be any desired compound having a secondary amino group, i.e., having a nitrogen atom substituted with a hydrogen atom and two organic groups. The secondary amine preferably has only one secondary amino group.

Preferred secondary amines have a molecular weight of no more than 1000 g/mol, more particularly no more than 500 g/mol, with a molecular weight of no more than 300 g/mol being particularly preferred. A very particularly preferred embodiment uses a secondary amine having a molecular weight of no more than 200 g/mol.

The secondary amine is preferably liquid or gaseous at standard conditions (20° C., 1 bar). In a particular embodiment, it is gaseous at standard conditions.

More particularly, the secondary amine is a compound having only one nitrogen atom, which nitrogen atom is substituted with a hydrogen atom and two hydrocarbon groups. The two hydrocarbon groups may be identical or different. It is preferable for hydrocarbon groups to be concerned which each independently have 1 to 10 carbon atoms. The two hydrocarbon groups may each independently be an aliphatic group, more particularly an alkyl group, an aromatic group, more particularly a phenyl group, or a group having aromatic and aliphatic portions; the latter is, for example, an alkylene group substituted with an aromatic group, or a phenyl group having aliphatic substituents.

In a preferred embodiment, the secondary amine is a dialkylamine of formula II

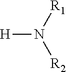

where R₁ and R₂ are each independently a C1 to C10 alkyl group.

Preferably. R1 and R2 are each independently a C1 to C4 alkyl group. It is particularly preferred for both R1 and R2 to be methyl groups.

In one particular embodiment, the process is used to prepare 2-chlorodialkylbenzylamines of the formula III

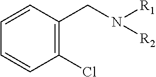

The starting compounds used therefor are an aldehyde of formula I, a dialkylamine of formula II and hydrogen.

It is particularly preferable to use the process to prepare 2-chlorodimethylbenzylamine (R₁ and R₂ in formulae II and III are then both methyl groups).

It is likely that 2-chlorobenzaldehyde and the dialkylamine react to form an intermediate in the reaction, in which case the corresponding aminal, hemiaminal or iminium ion, for example, may be formed. This intermediate is hydrogenated in the presence of hydrogen to form the 2-chloro-dialkylbenzylamine.

The Reaction

The carbonyl compound is reacted with the dialkylamine and hydrogen.

The reaction can be carried out with or without a solvent. The reaction may, for example, be carried out in the presence of a solvent for the carbonyl compound or for the dialkylamine. Useful solvents for the carbonyl compound, in particular for 2-chlorobenzaldehyde, include, for example, hydrophilic organic solvents. Hydrophilic aprotic organic solvents are particularly preferred. Examples include, for example, ethers, such as tetrahydrofuran, diethyl ether, methyl tertiarybutyl ether or 1,4-dioxane. Suitable solvents for the dialkylamine include water or hydrophilic organic solvents in which the dialkylamine is at least partially soluble. Examples include, in particular, water, ethers, such as tetrahydrofuran, or alcohols such as methanol or ethanol.

It is preferable not to use a solvent in the reaction.

The carbonyl compound, the dialkylamine, the hydrogen and any solvent used can be continuously supplied to the reactor separately or as a mixture of at least two of the starting materials mentioned. The starting materials are then commixed in a tubular reactor to form the reaction mixture.

Liquid starting materials, in particular, may be mixed beforehand and added to the reactor as a liquid mixture.

The reactor is preferably a tubular reactor.

Hydrogen is supplied in gaseous form, preferably by establishing and maintaining an appropriate hydrogen pressure. A mixture of hydrogen with inert gases such as nitrogen or noble gases may optionally also be used.

Gaseous dialkylamines may be supplied to the reactor together with the hydrogen gas. Dimethylamine, for example, has a boiling point of 7° C. The hydrogen stream may be contacted with the dimethylamine beforehand, such that the gas stream comprises sufficient dimethylamine.

The dialkylamine is preferably used in at least equimolar amounts, more particularly it is used in a molar excess based on the carbonyl compound.

More particularly, the molar ratio of dialkylamine to the carbonyl compound is from 1:1 to 50:1, more preferably from 2:1 to 20:1 and most preferably from 3:1 to 15:1.

The hydrogen is supplied in gaseous form in sufficient amounts, generally in a molar excess based on the carbonyl compound.

The reaction is carried out as a continuous operation, i.e., the starting materials are continuously supplied and the products obtained are continuously removed.

The reaction mixture comprising the carbonyl compound, the secondary amine and hydrogen is contacted first with an acidic fixed bed and with a fixed bed of hydrogenation catalyst (hereinafter referred to as a hydrogenation fixed bed) in the reactor which is preferably a tubular reactor.

The acidic fixed bed is a fixed bed of acidic solid particles.

Acidic solid particles are to be understood as meaning solid particles that are acidic overall. The acidic solid particles therefore comprise acidic compounds in an amount such that the solid particles are acidic overall. More particularly, solid particles are concerned which lower the pH when at least 10 g of solid particles are added to 100 g of neutral water (pH 7) irrespective of whether the solid particles are soluble, partially soluble or insoluble in water. Other typical methods may also be used to characterize the acidic properties; these include titration methods (e.g., using Hammett indicators), adsorption methods, spectroscopic methods and test reactions. The acidic fixed bed comprises the acidic solid particles or consists thereof. In addition to the acidic solid particles, the acidic fixed bed may comprise further components, for example inert materials such as fillers.

The acidic fixed bed preferably comprises hydrogenation catalysts only in small amounts, more particularly the acidic fixed bed comprises no hydrogenation catalysts. The acidic fixed bed more particularly comprises less than 10 wt %, more preferably less than 5 wt % and most preferably less than 1 wt % of hydrogenation catalysts, based on the total weight of the acidic fixed bed. The hydrogenation catalysts are, in particular, those defined and listed below.

In a preferred embodiment, the acidic fixed bed comprises at least 50 wt %, more particularly at least 80 wt % of the acidic solid particles, with an acidic fixed bed comprising at least 95 wt % of the acidic particles being particularly preferred. In a particular embodiment, the acidic fixed bed consists of 100 wt % of the acidic solid particles.

The acidic solid particles may have any desired shape. They may be present in the form of powder of granules but may also be made into any other desired shape, for example by appropriate pressing. Solid particles shaped as cylinders, beads, rings or tablets are useful. The solid particles may, for example, have a longest diameter of up to 50 mm.

The acidic solid particles preferably comprise at least 20 wt %, more particularly at least 50 wt %, of acidic compounds, with acidic solid particles comprising at least 80 wt % of acidic compounds being particularly preferred. The solid particles in addition to acidic compounds may comprise further compounds, for example non-acidic compounds as a support.

In a particularly preferred embodiment, the acidic solid particles consist exclusively of acidic compounds.

The acidic compounds may be Lewis acids or Brønsted acids. Preferred acidic compounds are acidic metal oxides, phosphates, tungstates, sulfates and organic acids or their salts.

Examples of acidic metal oxides are, in particular, titanium dioxide, zirconium dioxide, aluminum oxide, silicon dioxide or mixed oxides of aluminum and silicon (zeolites), and acidic argillaceous earths. Some of the aluminum and silicon atoms of zeolites may be replaced by other atoms, for example aluminum can be replaced by other trivalent metals.

Examples of suitable acidic organic compounds include, in particular, organic ion-exchange resins with acid groups, for example carboxylic acid groups or sulfonic acid groups. Sulfonized copolymers of styrene and divinylbenzene (e.g., the Lewatit brand from Lanxess, Amberlite from Rohm & Haas) are, for example, concerned.

In a particularly preferred embodiment, the acidic fixed bed comprises metal oxides, more particularly zirconium dioxide, as the acidic compound. In a very particularly preferred embodiment, the acidic fixed bed consists exclusively of metal oxides, more particularly zirconium dioxide.

The acidic fixed bed can be positioned in the reactor as desired. It is generally positioned such that the total reactant stream passes through the acidic fixed bed. The acidic fixed bed therefore generally fills the total flow cross-section of the reactor. The volume of the acidic fixed bed is preferably selected such that the space velocity of the carbonyl compound mass flow over the acidic fixed bed is 0.0005 to 500 kg/hour (h), more particularly 0.005 to 50 kg/h, of carbonyl compound per liter of the acidic fixed bed, with a space velocity of 0.05 to 50 kg/h of carbonyl compound per liter of the acidic fixed bed being particularly preferred. The volume of the acidic fixed bed is regarded as the total reactor volume occupied by the acidic fixed bed, including the cavities between the particles of the acidic fixed bed.

The reaction mixture is contacted with the hydrogenation fixed bed after being contacted with the acidic fixed bed. The hydrogenation catalyst is a solid; therefore a heterogeneous catalyst is concerned.

The hydrogenation fixed bed comprises the solid hydrogenation catalyst or consists thereof. The hydrogenation fixed bed in addition to the hydrogenation catalyst may contain further components, for example inert materials such as fillers.

In a preferred embodiment, the hydrogenation fixed bed comprises not less than 50 wt %, more particularly not less than 80 wt % of the hydrogenation catalyst, with a hydrogenation fixed bed comprising not less than 95 wt % of the hydrogenation catalyst being particularly preferred. In a particular embodiment, the hydrogenation fixed bed consists of 100 wt % of the hydrogenation catalyst.

The hydrogenation catalyst may be a customary heterogeneous hydrogenation catalyst in particle form.

Heterogeneous catalysts for hydrogenation are catalytically active elements or compounds; they may be present in particle form without a support (unsupported catalysts) or they may have been applied to a support, for example calcium carbonate, silicon oxide, zirconium dioxide or aluminum oxide (supported catalysts).

Preferred hydrogenation catalysts comprise active metals, either in elemental form or in the form of compounds, for example oxides. The catalysts often comprise mixtures of active metals. In the following, the term metal therefore encompasses elemental metals and also metals present in chemical compounds either in ionic form or in covalently bonded form. When oxides or as the case may be also other compounds of the active metals are used, a reduction of the oxides to the metals is effected, generally at higher temperatures and, in particular, in the presence of hydrogen. This may occur when the reaction commences or it may be carried out beforehand in a separate step.

Suitable hydrogenation catalysts include, for example, those comprising a metal of the groups IVb, Vb, VIb, VIIb, VIIIb, Ib or IIb.

In a preferred embodiment, the hydrogenation catalyst is a supported catalyst.

Suitable hydrogenation catalysts include, for example, catalysts comprising nickel, palladium, platinum, cobalt, rhodium, iridium, copper, manganese, tin or ruthenium.

Hydrogenation catalysts comprising at least one metal of the cobalt, nickel or copper group of the periodic table are preferred, more particularly hydrogenation catalysts comprising at least one metal selected from cobalt, rhodium, iridium, nickel, palladium, platinum or copper are preferred. More particularly, the content of the aforementioned metals of the cobalt, nickel or copper group in the catalyst amounts to a total of at least 5 wt %, more preferably at least 20 wt % and most preferably at least 50 wt %, based on the total weight of all active metals of the catalyst (for metal compounds, e.g., oxides, only the metal fraction is considered). In one particular embodiment, mixtures of the aforementioned metals are used. Further active metals that may be used along with the aforementioned metals include, for example, manganese, tin, ruthenium or else alkali metals and alkaline earth metals.

In a particularly preferred embodiment, the hydrogenation catalysts comprise cobalt, nickel, copper or mixtures of these three metals in total amounts of 5 wt %, more preferably at least 20 wt % and most preferably at least 50 wt %, based on the total weight of all active metals of the hydrogenation catalyst.

The aforementioned metals may be present in elemental form or as compounds, for example as oxides. Examples of catalysts comprising metals in elemental form are Raney nickel or Raney cobalt.

The hydrogenation fixed bed may be installed in the reactor such that it immediately downstream of the acidic fixed or, depending on the geometry of the apparatus, it is a defined distance downstream of the of the acidic fixed bed.

The hydrogenation fixed bed can be positioned in the reactor as desired. It is generally positioned such that the total reactant stream passes through the hydrogenation fixed bed. The hydrogenation fixed bed therefore generally fills the total flow cross-section of the reactor. The volume of the hydrogenation fixed bed is preferably selected such that the space velocity of the carbonyl compound mass flow over the hydrogenation fixed bed is 0.005 to 50 kg/hour (h), more particularly 0.05 to 50 kg/h, per liter of the hydrogenation fixed bed. The volume of the hydrogenation fixed bed is regarded as the total reactor volume occupied by the hydrogenation fixed bed, including the cavities between the particles of the hydrogenation fixed bed. The volume of the hydrogenation fixed bed may be greater or smaller than the volume of the acidic fixed bed.

The volume ratio of the acidic fixed bed to the hydrogenation fixed bed may be, for example, in the range from 0.05 to 50, preferably in the range from 0.1 to 10. More preferably, the volumes of the acidic fixed bed and the hydrogenation fixed bed may be identical or similar, which corresponds to a volume ratio in the range from 0.5 to 2, more specifically in the range from 0.8 to 1.25.

The reaction of the carbonyl compound with the secondary amine and hydrogen can be carried out, for example, at temperatures of from 20 to 200° C., preferably from 40 to 120° C., more preferably from 60 to 100° C. The reaction can be carried out, for example, at atmospheric pressure or at superatmospheric pressure. In a preferred embodiment, it is carried out at elevated pressure, for example, at pressure of from 1.1 to 200 bar, more particularly from 5 to 100 bar and with a pressure of from 40 to 100 bar being very particularly preferred. The pressure is preferably established using an appropriate pressure of the supplied hydrogen or gas mixture (e.g., hydrogen and inert gas).

The reaction product is continuously removed from the reaction zone once it has flowed through the hydrogenation fixed bed.

When water or a hydrophilic solvent is used for the second amine, the reaction product may be biphasic. For example when 2-chlorodimethylbenzylamine is prepared from the compounds of formulae I and II and a hydrophilic solvent is used, an organic phase is obtained comprising the 2-chlorodimethylbenzylamine, any unconverted 2-chlorobenzaldehyde and any by-products. The aqueous/hydrophilic phase comprises the solvent in which the secondary amine was dissolved (water or a hydrophilic solvent), unconverted secondary amine and any by-products soluble therein. The organic phase can be easily removed. The unconverted starting materials (2-chlorobenzaldehyde) can be purified or removed, for example, by distillation or rectification.

It is preferable not to use a solvent in the reaction. Only one organic homogeneous phase is therefore obtained as reaction product and this comprises the reaction product, for example 2-chlorodialkylbenzylamine, any unconverted starting materials, for example 2-chlorobenzaldehyde, and any by-products soluble therein. The secondary amine can be removed by distillation or rectification and, if desired, recycled into the reaction. Dimethylamine (boiling point 7° C.) is volatile at the relevant temperatures and can be removed particularly easily (degasification). The organic phase may optionally be further worked up/purified by distillation or rectification.

The process according to the invention can be used to obtain reductive amination products, more particularly 2-chlorodialkylbenzylamines, easily and economically. The process allows amines to be prepared by reductive amination with high yield and selectivity.

### EXAMPLES

### Comparative Example

### Continuous Amination of 2-Chlorobenzaldehyde with Dimethylamine without ZrO₂ Bed

A heated tubular reactor having a length of 200 cm and an interior diameter of about 3.2 cm was packed with a layer of glass balls and subsequently with 500 ml of a nickel-, cobalt- and copper-containing catalyst supported on ZrO₂. The reactor was subsequently topped up with a layer of glass balls.

Prior to the reaction, the catalyst was activated for 24 hours at 280° C. under a stream of 200 I/h of hydrogen at atmospheric pressure. The temperature was lowered to 75° C., the pressure was raised to 80 bar and then 2-chlorobenzaldehyde (100-300 g/h), dimethylamine (320-350 g/h) and hydrogen (60-150 standard liters/h) were charged. Samples were taken at regular intervals and analyzed by gas chromatography.

In further comparative examples, the amounts of 2-chlorobenzaldehyde and dimethylamine introduced into the reactor per unit time were varied (abbreviated in the table to “space velocity” in kg/liter per hour). The equivalent ratio of dimethylamine to chlorobenzaldehyde, the pressure and the temperature were also varied (see table).

### Inventive Example

### Continuous Amination of 2-Chlorobenzaldehyde with Dimethylamine with ZrO₂ Bed

The lower part of a heated tubular reactor with a length of 200 cm and an internal diameter of about 3.2 cm was packed with about 450 ml of ZrO₂ (3 mm extrudates). After packing with a nickel-, cobalt- and copper-containing catalyst supported on ZrO₂ (500 ml, 3×4 mm tablets), the remaining residual volume of the tubular reactor was topped up with glass balls.

Prior to the reaction, the catalyst was activated for 24 hours at 280° C. under a stream of 200 l/h of hydrogen at atmospheric pressure. The temperature was lowered to 75° C., the pressure was raised to 80 bar and then 2-chlorobenzaldehyde (100-300 g/h), dimethylamine (320-350 g/h) and hydrogen (60-150 standard liters/h) were charged. Samples were taken at regular intervals and analyzed by gas chromatography.

In further examples, the amounts of 2-chlorobenzaldehyde and dimethylamine introduced into the reactor per unit time were varied. Again, the equivalent ratio of dimethylamine to chlorobenzaldehyde, the pressure and the temperature were also varied (see table).

TABLE
Parameters and results of comparative and inventive examples

| | Comparative example | | | | |
|---|---|---|---|---|---|
| Space velocity | 0.2 | 0.2 | 0.3 | 0.4 | 0.5 |
| [kg/l*h] | | | | | |
| Temp. [° C.] | 60 | 70 | 60 | 60 | 70 |
| Equiv. DMA | 10 | 10 | 10 | 7 | 7 |
| (mol_{DMA}/mol_{oCIBA}) | | | | | |
| Pressure [bar] | 40 | 80 | 80 | 80 | 80 |
| Yield | 79.9 | 94.8 | 95.3 | 96.3 | 96.8 |
| without ZrO₂ bed | | | | | |
| | Inventive example | | | | |
| Space velocity | 0.2 | 0.2 | 0.3 | 0.4 | 0.5 |
| [kg/l*h] | | | | | |
| Temp. [° C.] | 60 | 70 | 75 | 75 | 75 |
| Equiv. DMA | 10 | 10 | 7.3 | 5.5 | 4.4 |
| (mol_{DMA}/mol_{oCIBA}) | | | | | |
| Pressure [bar] | 40 | 80 | 80 | 80 | 80 |
| Yield | 94.7 | 97.7 | 98.0 | 98.7 | 98.1 |
| with ZrO₂ bed | | | | | |

1: A process for effecting reductive amination by reacting a carbonyl compound with a secondary amine and hydrogen, wherein the reaction is carried out as a continuous operation and the reaction mixture comprising the carbonyl compound, the secondary amine and hydrogen is contacted first with an acidic fixed bed and then with a fixed bed of a hydrogenation catalyst.

2: The process according to claim 1, wherein the carbonyl compound is an aromatic ring system comprising 5 to 12 carbon atoms and substituted with an organic group having an aldehyde group and at least one optional substituent selected from the group consisting of an organic group and a halogen atom.

3: The process according to claim 1, wherein a 2-chlorobenzaldehyde of formula I 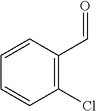
is used as the carbonyl compound.

4: The process according to claim 1, wherein the secondary amine is a dialkylamine.

5: The process according to claim 1, wherein the secondary amine is a dialkylamine of formula II 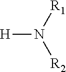
where R₁ and R₂ are each independently a C1 to C10 alkyl group.

6: The process according to claim 1, wherein a molar ratio of the carbonyl compound to the secondary amine is from 1:1 to 1:20.

7: The process according to claim 1, wherein the acidic fixed bed comprises titanium dioxide, zirconium dioxide, aluminum oxide, silicon dioxide or mixed oxides of aluminum and silicon (zeolites).

8: The process according to claim 1, wherein the acidic fixed bed comprises zirconium dioxide.

9: The process according to claim 1, wherein the hydrogenation catalyst comprises at least one metal selected from the group consisting of nickel, palladium, platinum, cobalt, rhodium, copper and iridium.

10: The process according to claim 1, wherein the hydrogenation catalyst is a supported catalyst.

11: The process according to claim 1, wherein a space velocity over the acidic fixed bed is from 0.0005 to 500 kg/h of carbonyl compound per liter of the acidic fixed bed.

12: The process according to claim 1, wherein a space velocity over the fixed bed of hydrogenation catalyst is from 0.005 to 50 kg/h of carbonyl compound per liter of the fixed bed of the hydrogenation catalyst.

13: The process according to claim 1, wherein a volume ratio of the acidic fixed bed to the fixed bed of the hydrogenation catalyst is in the range from 0.1 to 10.

14: The process according to claim 1 for preparing a 2-chlorodialkylbenzylamine of formula III 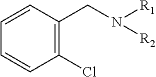
where both R₁ and R₂ are each independently a C1 to C10 alkyl group.

15: The process according to claim 1, wherein the acidic fixed bed consists of titanium dioxide, zirconium dioxide, aluminum oxide, silicon dioxide or mixed oxides of aluminum and silicon (zeolites).

16: The process according to claim 1, wherein the acidic fixed bed consists of zirconium dioxide.
